(19) United States
(12) Patent Application Publication
(10) Pub. No.: US 2021/0121682 A1
(43) Pub. Date: Apr. 29, 2021

(54) SOCK THAT WIRELESSLY DELIVERS ELECTRICAL SIGNALS DIRECTLY TO THE FOOT AND ANKLE MUSCLES FOR THE TREATMENT OF PAIN

(71) Applicant: Texas Woman's University, Denton, TX (US)

(72) Inventor: Ngozi D. Mbue, Rosharon, TX (US)

(21) Appl. No.: 17/060,857

(22) Filed: Oct. 1, 2020

Related U.S. Application Data
(60) Provisional application No. 62/926,508, filed on Oct. 27, 2019.

(51) Int. Cl. A61N 1/04; A61N 1/36; A61F 13/08; G16H 40/67; G16H 20/30; G06F 1/26; H04W 76/14; H04Q 9/00

(52) U.S. Cl. CPC A61N 1/0484; A61N 1/0456; A61N 1/36003; A61N 1/36034; A61F 13/08; A41B 11/00; G16H 20/30; G06F 1/26; H04W 76/14; H04Q 9/00; G16H 40/67

(57) ABSTRACT

Described herein is a sock that delivers electrical impulses to the muscles within the foot for reducing chronic pain and inflammation. The sock is removable, reusable and the electrical components that deliver the electrical impulses are integrated into the sock.

### PRIORITY CLAIM

This application claims priority to U.S. Provisional Application Ser. No. 62/926,508 entitled “SOCK THAT WIRELESSLY DELIVERS ELECTRICAL SIGNALS DIRECTLY TO THE FOOT AND ANKLE MUSCLES FOR THE TREATMENT OF PAIN” filed Oct. 27, 2019, which is incorporated herein by reference in its entirety.

### STATEMENT REGARDING FEDERALLY SPONSORED RESEARCH OR DEVELOPMENT

This invention was made with government support under National Science Foundation (Award ID 1954004)-Federal (CFDA #47.041 Engineering Grants). The government has certain rights in the invention.

### BACKGROUND OF THE INVENTION

### 1. Field of the Invention

The invention generally relates to a nerve stimulating sock for reducing pain and promoting muscle recovery in the feet of subjects.

### 2. Description of the Relevant Art

Nurses, and other professionals, are at high risk for developing chronic musculoskeletal pain due to the high physical demands such as the lifting of patients and prolonged standing. Chronic foot pain can cause disability and loss of motivation while being a major contributor to sick leave and early retirement throughout the profession. It is also causing a shortage of nursing staff that is imposing pressure on the entire healthcare system. According to a recent report, chronic and disabling foot pain afflicts over 50 percent of nurses. Rates are even higher in nurses who are obese and have been working for over 40 years. Intervention commonly consists of non-steroidal anti-inflammatory drugs (ibuprofen), steroids (cortisone) and plasma injections. Non-pharmacologic treatments involve the use of splints, special footwear, compression socks and orthotic inserts. While these treatments can have various degrees of efficacy, they fail to treat the underlying cause of pain. Hence, there is a significant unmet need for new treatment options for nurses and those in similar professions that suffer from chronic foot and ankle pain.

Several recent studies suggest the use of electrical nerve stimulation (Electrical Muscle Stimulation (EMS)/Transcutaneous Electrical Nerve Stimulation (TENS)) may help reduce chronic pain across several different indications. As a result, they have spawned the development and FDA approval of numerous commercial devices that reduce chronic pain with electrical stimulation. However, there are limited neuro stimulating devices specifically aimed for the treatment of chronic foot and ankle pain.

### SUMMARY OF THE INVENTION

Described herein is a sock that wirelessly delivers electrical stimulation directly to the foot and ankle muscles for the treatment of pain. The sock, during use, will provide pain relief for people suffering from chronic foot and ankle pain without the need for medications.

In one embodiment, a sock delivers electrical stimulation to the muscles within the foot and ankle when an individual experiences pain. A removable and reusable electrical stimulator is integrated into the plantar area of the sock. When pain is detected, nerve stimulation can be activated by the user through a remote-control device (e.g., a hand-held control device of a smartphone). In addition, the electrical stimulation can be automatically delivered if the user is in the standing position for an extended period. The electrical stimulation can be used multiple times a day (e.g., three to four times a day). In some embodiments, electrical simulation may be used for up to 60 minutes per day.

In an embodiment, a sock for providing relief from foot pain is composed of a tubular flexible fabric. The sock includes a transcutaneous electrical nerve stimulation (TENS) device removably attached to an inner surface of the tubular flexible fabric. The TENS device includes one or more TENS electrodes, a TENS power supply coupled to the TENS electrodes, and a TENS controller configured to control the supply of power from the TENS power supply to the TENS electrodes. The TENS controller is further configured to receive wireless control signals from a remote-control device. The TENS device is placed in the tubular flexible fabric such that the TENS electrodes contact the foot of a user when the sock is placed on the user's foot. The TENS power supply may be a rechargeable battery. The TENS controller may include a radio transceiver to receive control signals and send information to a remote-control device.

The TENS device applies electrical pulses to the foot through the one or more TENS electrodes, wherein the electrical pulses have a frequency of between 2 and 150 Hz, and wherein the delay time between electrical pulses is between 10 μs to 500 μs, and wherein the electrical pulses are applied at a current of 1 mA to 100 mA.

In one embodiment, the sock is configured as a compression sock, wherein the pressure applied by the sock is between 10 mmHg and 50 mmHg. The compression sock may be a gradient pressure sock which applies a higher pressure to an ankle region of the user than the pressure applied to the calf region of the user.

The sock may further include an electrical muscle stimulation (EMS) device removably attached to an inner surface of the tubular flexible fabric. The EMS device includes one or more EMS electrodes, an EMS power supply coupled to the EMS electrodes, and a EMS controller configured to control the supply of power from the EMS power supply to the EMS electrodes. The EMS controller is further configured to receive wireless control signals from the remote-control device. The EMS device is placed in the tubular flexible fabric such that the EMS electrodes contact the user at or proximate to the ankle of the user when the sock is placed on the user's foot.

The sock may also include an insole coupled to the tubular flexible fabric.

Activation of the TENS and/or EMS units may be controlled by a remote-control device. The remote-control device may be a custom built remote, or may be embodied in an application that may be installed on a smartphone.

In an embodiment, a method of providing transcutaneous electrical nerve stimulation to a foot of a user includes: placing a sock on a user's foot, the sock composed of a tubular flexible fabric, wherein the sock comprises a transcutaneous electrical nerve stimulation (TENS) device removably attached to an inner surface of the tubular flexible fabric, the TENS device comprising one or more TENS electrodes, a TENS power supply coupled to the TENS electrodes, and a TENS controller configured to control the supply of power from the TENS power supply to the TENS electrodes, wherein the TENS controller is further configured to receive wireless control signals from a remote-control device; positioning the sock on the user's foot such that the TENS electrodes contact the foot of a user; and remotely activating the TENS device to initiate the application of electrical pulses through the TENS electrodes into the foot. The method further includes removing the sock from the user's foot; removing the TENS device from the sock; and recharging the power supply of the TENS device.

In embodiments in which the sock includes an EMS electrode, the method includes positioning the sock on the user's foot such that the EMS electrodes contact the user at or proximate to the ankle of the user when the sock is placed on the user's foot, and remotely activating the EMS device to initiate the application of electrical pulses through the EMS electrodes into the ankle region of the user. In some embodiments, the method includes activating the TENS device and the EMS device at different times. The method also includes activating the TENS device and the EMS device to produce electrical pulses such that the frequency and/or duration time and/or the current of the electrical pulses applied by the TMS device are different from the frequencies and/or duration time and/or the current of the electrical pulses applied by the EMS device.

In some embodiments, activation and control of the TENS and/or EMS devices may be controlled by a smartphone. In such embodiments, the method also includes: installing an application on a smartphone, wherein the application provides a graphic user interface that allows the user to activate the TENS device; and creating a wireless connection between the smartphone and the TENS controller. In an embodiment, the application automatically activates the TENS device at predetermined times. The predetermined times are selected by the user using the graphic user interface of the application.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a view of a sock that includes one or more TENS devices; and

FIG. 2 depicts a schematic diagram of a TENS device.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a,” “an,” and “the,” include singular and plural referents unless the content clearly dictates otherwise. Furthermore, the word “may,” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In an embodiment, a sock delivers electrical impulses to the muscles and nerves within the user's foot for reducing chronic pain and inflammation. The sock has electrical components integrated within the plantar area of the sock. When the user experiences pain, nerve stimulation is remotely activated (e.g., via their smartphone) and can provide instant pain relief. The user can also use a remote-control device (e.g., their smartphone) to automatically deliver neurostimulation if they expect to be standing for long periods or when sleeping at night. In some embodiments, in a single day, the device allows the user to activate stimulation three to four times a day for a total of 60 minutes of nerve stimulation. The sock also features a shock-absorbing insole. The sock may also be a compression sock that includes the electrical components.

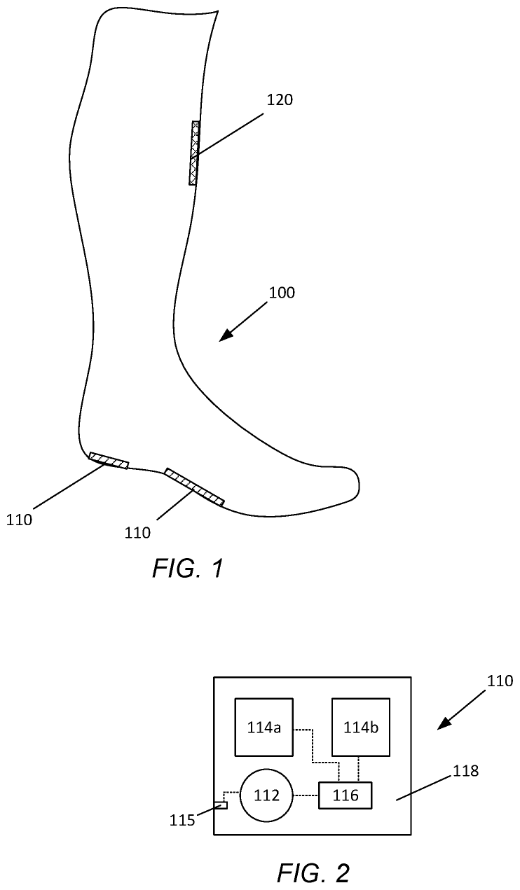

FIG. 1 depicts a schematic view of a sock 100 having one or more TENS devices 110. The sock may be composed of any tubular flexible fabric. As is generally know, the sock includes an open end and a closed end that allows a user of the sock to insert their foot into the sock. The sock has an outer surface and an inner surface, the inner surface being generally disposed against the foot and the leg of the user when the sock is in use.

One or more transcutaneous electrical nerve stimulation (TENS) devices 110 are removably attached to the sock. The TENS devices may be coupled, for example, using a hook-loop system. When using a hook-loop system, one component of the hook-loop system may be attached to the TENS device using an adhesive. The complementary portion of the hook-loop system may be attached to the inner surface of the sock using an adhesive or may be sewn into the flexible fabric. Other methods of removable attachment are contemplated such as a button attachment or a pouch into which the TENS device may be placed and removed.

FIG. 2 depicts a schematic diagram of a TENS device 110. TENS device 110 may include a TENS power supply 112, a TENS electrode 114, and a TENS controller 116. Generally, a TENS device stimulates nerves by passing an electrical current through the skin. For nerve stimulation, the electric current may be applied as electric pulses. As shown in FIG. 2, a TENS power supply 112 (e.g., a rechargeable battery) is incorporated into the TENS device to provide power to the TENS electrodes. The use of a power supply that is incorporated into the TENS device allows the TENS device to be used without needing any external wires. The TENS device may include a charging port 115 which is electrically coupled to the TENS power supply 112.

TENS controller 116 couples the TENS power supply 112 to the TENS electrodes 114a and 114b. The controller acts as a switch between the TENS power supply and the TENS electrodes. The controller includes a processor, memory, and a transceiver. The processor and memory allow control signals to be processed and activated by the controller. The transceiver allows wireless communication between the controller and a remote-control device. The wireless communication between the controller and a remote-control device may be through radio frequency communication (e.g., Bluetooth).

The TENS device includes at least two TENS electrodes 114a, 114b (or at least one electrode pair). The electrodes are placed on the outer surface of the TENS device, such that the electrodes come into contact with the user's foot when the sock is being used. The electrodes are preferably spaced apart to create a conductive path through the user's skin, such that an electric current passes through the skin from one electrode to the other electrode.

The body of TENS device 110 preferably has a low thickness to enable the device to be comfortably worn inside the sock and in contact with the patient's foot. In some embodiment, TENS device 110 may have a thickness of between about 1 mm and 10 mm. The TENS device may be formed from a soft material. For examples, the body of TENS device 110 may be formed from a cloth material. In some embodiments, the body of the TENS device is formed from the same flexible fabric used for the sock. The interior of the TENS device may include a foam material. The foam material may provide support for the electrical components (electrodes, power supply and controller) within the body. The foam may also provide a cushioning effect between the TENS device and the user's foot. The combination of a thin profile and a soft casing allows the TENS device to be comfortably placed in the sock during use, even when the user is wearing shoes.

To reduce pain in the foot, the electrodes are preferably placed in the plantar fasciitis region of the foot. The proper placement of the electrodes can be ensured by placing the TENS device in the proper location inside the sock. Thus, when the sock is properly placed on the user's foot, the TENS device will be positioned against the plantar fasciitis region of the foot.

The electric current is preferably applied as pulses of electricity. The rate at which the pulses are applied may be measured in pulses per second (Hz). For nerve stimulation, the pulse rate may range from 2 Hz to 150 HZ. Preferably, the pulse rate is between 40 Hz and 150 Hz, more preferably the pulse rate is between 80 Hz and 120 Hz. The time between each pulse can affect the efficacy of the treatment.

For treatments using TENS, the purpose is to stimulate the nerves to reduce the sensation of pain in the effected area. The application of electrical stimulation can also cause muscle contraction. It is preferred, for pain suppression, that the electrical current is applied from the TENS device such that the sensation of pain is reduced without causing significant muscle contractions. One way that this may be accomplished is by adjusting the delay time between pulses. The delay time between pulses may be varied from 10 μs to 500 μs. Generally, to reduce muscle contraction, shorter delay times are used. For TENS devices, a delay time of between 10 μs and 100 μs is used, preferably between 10 μs and 50 μs is used. Longer time delays, for example time delays greater than 100 μs, tend to produce muscles contractions.

Another parameter of the electrical stimulation is the current applied by each pulse. The stronger the current, the more effect the electrical stimulation will have. The current should, however, not be too strong, as strong currents can be uncomfortable to the user and can, inadvertently harm the user's skin. For TEMS treatments, the applied current should be between 1 mA and 100 mA, preferably between 10 mA and 50 mA.

Another parameter of the electrical stimulation is the duration of the application of the electrical stimulation. The duration of the electrical stimulation ranges from 10 to 60 minutes, with typical application times of 30 minutes.

Since the TENS device includes a power source, electrodes, and a controller, the TENS device does not require a wired connection. To operate the TENS device, a wireless remote-control device is used. The wireless remote-control device includes a transmitter or transceiver that can be used to communicate with the controller through the controller transceiver. During use, a connection between the TENS device and the remote-control device may be established. The remote-device may be used to activate the TENS device, which, upon receipt of the activation signal, begins providing electrical pulses through the TENS electrodes. The remote-control device may also be used to set the operating parameters (e.g., pulse frequency, delay time, current and duration) of the TENS device.

In one embodiment, the remote-control device is provided with the sock as part of a pain relief system. Alternatively, a smartphone may be used as a remote-control device. The smartphone may include an application (e.g., software installed by the user onto the smartphone) that allows the smartphone to communicate with the TENS controller. The application provides a graphic user interface that allows the user to activate the TENS device and/or alter the operating parameters of the TENS device.

The remote-control device may also be programmed to activate the TENS device at predetermined times. For example, a user of the device may create a schedule that activates the TENS device at predetermined times while the user is working. The user can, therefore, create a proactive schedule which may reduce foot pain and/or prevent foot pain from occurring. Using a smartphone as the remote-control device, the graphic user interface of the smartphone application can provide options for setting automatic activation of the TENS device.

In some embodiments, an Electrical Muscle Stimulation (EMS) device 120 may also be incorporated into the sock. The EMS device has the same configuration as the TENS device depicted in FIG. 2. In some embodiments, the TENS device depicted in FIG. 2 may also be used as an EMS device. EMS device 120 may include a EMS power supply, an EMS electrode, and a EMS controller. Similar to the TENS device, an EMS device passes an electrical current through the skin. The effect of an EMS device is different from the TENS device. An EMS device uses electrical pulses to create muscle contractions. Muscle contractions can help promote increased blood flow through the legs into the foot. This increase blood flow can help promote healing in the foot. For nerve stimulation, the electric current of an EMS may also be applied as electric pulses. An EMS power supply (e.g., a rechargeable battery) is incorporated into the EMS device to provide power to the EMS electrodes. The use of a power supply that is incorporated into the EMS device allows the EMS device to be used without needing any external wires. The EMS device may include a charging port which is electrically coupled to the EMS power supply.

Similar to the TENS device, the EMS controller couples the EMS power supply to two (or more) EMS electrodes. The controller acts as a switch between the EMS power supply and the EMS electrodes. The controller includes a processor, memory, and a transceiver. The processor and memory allow control signals to be processed and activated by the controller. The transceiver allows wireless communication between the controller and a remote-control device. The wireless communication between the controller and a remote-control device may be through radio frequency communication (e.g., Bluetooth).

The EMS device includes at least two EMS electrodes (or at least one electrode pair). The electrodes are placed on the outer surface of the EMS device, such that the electrodes come into contact with the user's leg (typically the calf) when the sock is being used. The electrodes are preferably spaced apart to create a conductive path through the user's skin, such that an electric current passes through the skin from one electrode to the other electrode.

Again, similar to the TENS device, the EMS device 120 preferably has a low thickness to enable the device to be comfortably worn inside the sock and in contact with the patient's leg. In some embodiments, EMS device 120 may have a thickness of between about 1 mm and 10 mm. The EMS device may be formed from a soft material. For examples, the body of EMS device 120 may be formed from a cloth material. In some embodiments, the body of the EMS device is formed from the same flexible fabric used for the sock. The interior of the EMS device may include a foam material. The foam material may provide support for the electrical components (electrodes, power supply and controller) within the body. The foam may also provide a cushioning effect between the EMS device and the user's leg. The combination of a thin profile and a soft casing allows the EMS device to be comfortably placed in the sock during use.

To promote blood flow into the foot, the electrodes are preferably placed on the calf. The proper placement of the electrodes can be ensured by placing the EMS device in the proper location inside the sock. Thus, when the sock is properly placed on the user's foot, the EMS device will be positioned against the calf of the user.

The electric current is preferably applied as pulses of electricity. The rate at which the pulses are applied may be measured in pulses per second (Hz). For muscle contraction to promote blood flow to the foot, the pulse rate may range from 2 Hz to 150 HZ. Preferably, the pulse rate is between 10 Hz and 100 Hz, more preferably the pulse rate is between 30 Hz and 70 Hz.

For treatments using EMS, the application of electrical stimulation is used to cause muscle contractions that promote blood flow to the foot. One way that this may be accomplished is by adjusting the delay time between pulses. The delay time between pulses may be varied from 10 μs to 500 μs. Generally, to cause muscle contraction, longer delay times are used. For EMS devices, a delay time of between 100 μs and 500 μs is used, preferably between 200 μs and 300 μs is used.

Another parameter of EMS is the current applied by each pulse. The stronger the current, the more effect the electrical stimulation will have. The current should, however, not be too strong, as strong currents can be uncomfortable to the user and can, inadvertently harm the user's skin. For EMS treatments, the applied current should be between 1 mA and 100 mA, preferably between 10 mA and 50 mA.

Another parameter of the electrical stimulation is the duration of the application of the electrical stimulation. The duration of the electrical stimulation for EMS ranges from 10 to 60 minutes, with typical application times of 30 minutes.

The remote-control device, provided with the sock as part of a pain relief system. Alternatively, a smartphone may be used as a remote-control device. The smartphone may include an application (e.g., software installed by the user onto the smartphone) that allows the smartphone to communicate with both the TENS controller and the EMS controller. The application provides a graphic user interface that allows the user to activate the TENS device and the EMS device and/or alter the operating parameters of the TENS device and the EMS device. The remote-control device also allows each device to be independently operated and activated. This allows the TENS device and the EMS device to be activated at different times.

In some embodiments, the sock may provide compression to the leg and foot. Compression counters dependent edema, which can contribute to foot pain (tight shoes). Compression will help prevent blood pooling and vascular problems, while the accompanying targeted and pulsating EMS treatment will allow for plantar muscles conditioning and better arterial flow to the foot. Use of EMS and compression to promote blood flow through the foot may promote plantar wound healing.

In an embodiment, the sock is formed from a flexible fabric that imparts compression to the foot and/or leg. The compression material may apply between 8 mmHg up to 50 mmHg of compression to the foot and/or leg. The compression material preferably applies a gradient pressure to the foot and leg. A gradient pressure is created by altering the fabric such that the pressure applied to the foot and leg varies. For improving foot pain, the fabric of the sock may be adjusted such that a higher pressure is applied to the ankle region of the user than the pressure applied to the calf region of the user.

In an embodiment, a shock absorbing insole is integrated in the plantar region of the sock. In an optional embodiment, a closing system is used to secure the sock to the user's leg. A closing system, in some embodiments, includes, but is not limited to, a clutch reel system or one or more straps.

While the use of electrical stimulation, including over counter products and TENS system, has been widely prescribed for reducing pain, there does not appear to be wearable socks with embedded electrical stimulation component for facilitating smart delivery of daily plantar electrical stimulation during activities of daily living. In addition, none of the currently available compression socks appear to include electrical stimulation and ease of wearing components, essential to improve acceptability and effectiveness to reduce foot and back pain among users.

The socks described herein are capable of being worn continuously. For example, the sock may be worn continuous while the user is wearing shoes. The sock, in this case, acts as a normal sock, with the additional features mentioned above. The sock also has the potential to wear during sleep and adjusted based on intensity of plantar pain thereby, alleviating pain upon awakening.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A sock composed of a tubular flexible fabric, the sock comprising:
a transcutaneous electrical nerve stimulation (TENS) device removably attached to an inner surface of the tubular flexible fabric, the TENS device comprising one or more TENS electrodes, a TENS power supply coupled to the TENS electrodes, and a TENS controller configured to control the supply of power from the TENS power supply to the TENS electrodes, wherein the TENS controller is further configured to receive wireless control signals from a remote-control device;
wherein the TENS device is placed in the tubular flexible fabric such that the TENS electrodes contact the foot of a user when the sock is placed on the user's foot.

2. The sock of claim 1, wherein the TENS device applies electrical pulses to the foot through the one or more TENS electrodes, wherein the electrical pulses have a frequency of between 2 and 150 Hz, and wherein the delay time between electrical pulses is between 10 μs to 500 μs, and wherein the electrical pulses are applied at a current of 1 mA to 100 mA.

3. The sock of claim 1, wherein the sock is configured as a compression sock, wherein the pressure applied by the sock is between 10 mmHg and 50 mmHg.

4. The sock of claim 1, wherein the compression sock is a gradient pressure sock which applies a higher pressure to an ankle region of the user than the pressure applied to the calf region of the user.

5. The sock of claim 1, further comprising an electrical muscle stimulation (EMS) device removably attached to an inner surface of the tubular flexible fabric, the EMS device comprising one or more EMS electrodes, an EMS power supply coupled to the EMS electrodes, and a EMS controller configured to control the supply of power from the EMS power supply to the EMS electrodes, wherein the EMS controller is further configured to receive wireless control signals from the remote-control device, wherein the EMS device is placed in the tubular flexible fabric such that the EMS electrodes contact the user at or proximate to the ankle of the user when the sock is placed on the user's foot.

6. The sock of claim 1, further comprising an insole coupled to the tubular flexible fabric.

7. The sock of claim 1, wherein the remote-control device is a smartphone.

8. The sock of claim 1, wherein the power supply is a rechargeable battery.

9. The sock of claim 1, wherein the controller comprises a radio frequency transceiver.

10. A method of providing transcutaneous electrical nerve stimulation to a foot of a user comprising:
placing a sock on a user's foot, the sock composed of a tubular flexible fabric, wherein the sock comprises a transcutaneous electrical nerve stimulation (TENS) device removably attached to an inner surface of the tubular flexible fabric, the TENS device comprising one or more TENS electrodes, a TENS power supply coupled to the TENS electrodes, and a TENS controller configured to control the supply of power from the TENS power supply to the TENS electrodes, wherein the TENS controller is further configured to receive wireless control signals from a remote-control device;
positioning the sock on the user's foot such that the TENS electrodes contact the foot of a user; and
remotely activating the TENS device to initiate the application of electrical pulses through the TENS electrodes into the foot.

11. The method of claim 10, wherein the electrical pulses have a frequency of between 2 and 150 Hz, and wherein the delay time between electrical pulses is between 10 μs to 500 μs, and wherein the electrical pulses are applied at a current of 1 mA to 100 mA.

12. The method of claim 10, further comprising:
removing the sock from the user's foot;
removing the TENS device from the sock; and
recharging the power supply of the TENS device.

13. The method of claim 10, wherein the sock further comprises:
an electrical muscle stimulation (EMS) device removably attached to an inner surface of the tubular flexible fabric, the EMS device comprising one or more EMS electrodes, an EMS power supply coupled to the EMS electrodes, and an EMS controller configured to control the supply of power from the power supply to the EMS electrodes, wherein the EMS controller is further configured to receive wireless control signals from the remote-control device,
wherein the method further comprises:
positioning the sock on the user's foot such that the EMS electrodes contact the user at or proximate to the ankle of the user when the sock is placed on the user's foot, and
remotely activating the EMS device to initiate the application of electrical pulses through the EMS electrodes into the ankle region of the user.

14. The method of claim 13, further comprising activating the TENS device and the EMS device at different times.

15. The method of claim 13, further comprising activating the TENS device and the EMS device to produce electrical pulses such that the frequency and/or duration time and/or the current of the electrical pulses applied by the TMS device are different from the frequencies and/or duration time and/or the current of the electrical pulses applied by the EMS device.

16. The method of claim 10, further comprising:
installing an application on a smartphone, wherein the application provides a graphic user interface that allows the user to activate the TENS device; and
creating a wireless connection between the smartphone and the TENS controller.

17. The method of claim 16, wherein the application automatically activates the TENS device at predetermined times.

18. The method of claim 17, wherein the predetermined times are selected by the user using the graphic user interface of the application.
